(19) United States
(12) Patent Application Publication
(10) Pub. No.: US 2014/0162958 A1
(43) Pub. Date: Jun. 12, 2014

(54) Copolymer-1 Composition and Methods of Use

(71) Applicant: Michael J. Robbins, North Branford, CT (US)

(72) Inventor: Michael J. Robbins, North Branford, CT (US)

(21) Appl. No.: 13/712,521

(22) Filed: Dec. 12, 2012

(51) Int. Cl. A61K 38/16; A61K 31/445

(52) U.S. Cl. CPC A61K 38/16; A61K 31/445
USPC 514/17.9; 514/1.1

(57) ABSTRACT

Disclosed herein are compositions that comprise copolymer-1 and an anesthetic agent. Also provided are methods that include the administration of a composition comprising copolymer-1 and an anesthetic agent. Such methods are useful for the treatment of multiple sclerosis; for reducing the occurrence and/or severity of the side effects associated with a therapeutic regimen comprising administration of copolymer-1; and for increasing patient compliance with a therapeutic regimen comprising administration of copolymer-1.

### FIELD

The disclosure relates to compositions comprising copolymer-1 in combination with an amount of an anesthetic agent, as well as methods encompassing the use of such compositions.

### BACKGROUND

Copolymer-1, or glatiramer, is a synthetic polypeptide analog of myelin basic protein (MBP), which is a natural component of the myelin sheath. Interest in copolymer-1 as an immunotherapy for multiple sclerosis stems from observations first made in the 1950s that myelin components such as MBP prevent or arrest experimental autoimmune encephalomyelitis (EAE). EAE is a disease resembling multiple sclerosis that can be induced in susceptible animals.

Copolymer-1 was developed by Drs. Sela, Arnon, and their co-workers at the Weizmann Institute (Rehovot, Israel). It was shown to suppress EAE (Eur. J. Immunol. (1971) 1:242; U.S. Pat. No. 3,849,550). More recently, copolymer-1 was shown to be beneficial for patients with the exacerbating-remitting form of multiple sclerosis (N. Engl. J. Med. (1987) 317:408). Patients treated with daily injections of copolymer-1 had fewer exacerbations and smaller increases in their disability status than control patients.

Copolymer-1 is a mixture of polypeptides composed of alanine, glutamic acid, lysine, and tyrosine in a molar ratio of approximately 6:2:5:1, respectively. It can be synthesized by chemically polymerizing the four amino acids, forming products with average molecular weights ranging from 4,000 to over 40,000 daltons (see, e.g., U.S. Pat. No. 3,849,550). In recent years, Teva Pharmaceuticals has marketed copolymer-1, or glatiramer acetate, under the name COPAXONE® (alternatively herein, the “Drug”) as a self-administered injection for the treatment of multiple sclerosis. The Drug has been approved by the Food and Drug Administration (FDA) for reducing the frequency of disease relapses (Relapsing-Remitting Multiple Sclerosis, RRMS), but not for reducing the progression of disability. Observational studies, but not randomized controlled trials, suggest that the drug may reduce progression of disability.

To date, COPAXONE® has been approved for marketing in 49 countries worldwide, including the United States, Israel, Canada and 24 European Union Countries. Approval in the U.S. was obtained in 1996 and in the U.K. in August 2000.

While the Drug provides a clinical benefit to patients suffering from multiple sclerosis, it is also associated with a number of side effects that can create problems with patient compliance and continuation of the therapy. For example, local injection site reactions, (“LISRs” or “Site Reactions”) are common and persistent for many patients who require the daily administration of COPAXONE®. Such Site Reactions can manifest as immediate and/or delayed reactions typically arising within about 5 minutes and lasting up to 24 hours or longer. The Site Reactions may often include pain, erythema, edema, cutaneous hypersensitivity, and/or inflammation, among others. Discontinuation of therapy due to pain regularly occurs in clinical practice and the manufacturer's only recommended solutions to the problem involve rotating the injection site amongst various specified body regions (including the arms, thighs, abdominal wall and buttocks), application of ice or ice packs, and use of oral analgesics (e.g., ibuprofen, etc.). Teva also provides for nursing education to patients on managing injection site pain. (See, Product Monograph).

Other strategies to reduce the number, frequency, and/or severity of Site Reactions have been developed in an effort to promote patient compliance and improve quality of life; however Site Reactions continue to pose significant problems for a large number of patients who use the Drug. These strategies include attempts to formulate alternative glatiramer acetate compositions focused on reducing the injection volume (<1.0 mL) or reducing injection frequency (higher glatiramer acetate concentrations), which have been evaluated in clinical studies.

For example, a Phase III GALA (Glatiramer Acetate Low-Frequency Administration) clinical trial conducted by Teva assessed the efficacy, safety, and tolerability of 40 mg/mL dose glatiramer acetate subcutaneous injection (versus placebo) three times a week in RRMS patients. While the results of the study showed that the high dose formulation administered three times a week met its primary endpoint (reducing the annualized relapse rate relative to placebo), patients in the study reported occurrence of reactions, including injection site reactions, headaches, and nasopharyngitis.

Teva also conducted a study that assessed a lower-volume injection of COPAXONE®. The SONG study (‘Study of New Glatiramer Acetate Formulation’) explored the safety and tolerability of a 20 mg/0.5 mL injection of COPAXONE® versus the approved formulation of 20 mg/1.0 mL. Both formulations were safe and well tolerated, with a percentage of patients receiving the lower-volume injection reporting less pain immediately after and at five minutes after injection, as well as fewer or less severe Site Reactions at five minutes and at 24 hours post-injection. Adverse events were reported by 12.5 percent of patients when injecting 20 mg/1.0 mL and by 18.1 percent of patients when injecting 20 mg/0.5 mL. Overall, the adverse event profile of either formulation in this study was consistent with the observed safety profile of COPAXONE® in placebo-controlled trials and post-marketing experience. (See, Murray, R., et al., International TMS Care, (May 2010) 12(Suppl.1):54.).

The development of desensitization protocols has also been suggested as a possible option for patients that experience COPAXONE®-associated Site Reactions. In one study a 4-hour outpatient procedure involved an initial 20 ng subcutaneous injection of glatiramer acetate suspension followed by subcutaneous injections every 15 minutes with increased dosages of the suspension. While not conclusive, the results suggested that patients having Site Reactions in response to COPAXONE® therapy may be able to be successfully and safely desensitized to it, and resume recommended use.

Attempts to reformulate glatiramer acetate for administration to patients by routes other than subcutaneous injection (e.g., nasal administration) have not been successful.

Accordingly, while some strategies that are in current development show potential for reducing Site Reactions, a need still exists for a composition that provides for the clinical benefit of copolymer-1 (e.g., glatiramer acetate or COPAXONE®) while reducing the number, frequency, and/or severity of the Site Reactions associated with copolymer-1 therapy. Such compositions and methods would provide a benefit for improving patient compliance and quality of life for those who can benefit from using the Drug.

### SUMMARY

In an aspect, the disclosure provides a composition comprising copolymer-1, an anesthetic, and a carrier, diluent, or vehicle. In embodiments the anesthetic comprises a local anesthetic. In some embodiments, the composition is formulated as an injectable dosage form. In some embodiments the local anesthetic is selected from the group consisting of bupivacaine, ropivacaine, lidocaine, chloroprocaine, and mepivacaine. In some embodiments the local anesthetic is selected from lidocaine, chloroprocaine, and mepivacaine. In some embodiments the local anesthetic comprises bupivacaine. In some embodiments the local anesthetic comprises ropivacaine. In further embodiments, the composition comprises a total amount of bupivacaine or ropivacaine of about 0.01% to about 1.0% (w/v). In some embodiments, the composition comprises a total amount of lidocaine, chloroprocaine, or mepivacaine of about 0.1% to about 1.0% (w/v). In embodiments the copolymer-1 comprises injectable glatiramer acetate having an average molecular weight of about 4 kilodaltons to about 9 kilodaltons. In further embodiments the copolymer-1 comprises COPAXONE®.

In another aspect, the disclosure provides a method for treating multiple sclerosis comprising administering to a subject in need thereof a composition comprising copolymer-1, an anesthetic, and a carrier, diluent, or vehicle. In some embodiments, the method comprises administering the composition by injection.

In another aspect, the disclosure provides a method for reducing a Site Reaction associated with administration of copolymer-1 to a subject, comprising administering to a subject in need thereof a composition comprising copolymer-1, an anesthetic, and a carrier, diluent, or vehicle. In some embodiments, the Site Reaction is associated with administration of copolymer-1 by injection. In embodiments, the method comprises administering the composition by injection.

In a further aspect, the disclosure provides a method for increasing patient compliance with a therapeutic regimen that comprises administration of copolymer-1, wherein the method comprises administering to the patient a composition comprising copolymer-1, an anesthetic, and a carrier, diluent, or vehicle. In some embodiments, the therapeutic regimen comprises an injectable dosage form of copolymer-1. In some embodiments, the method comprises administering the composition by injection.

The disclosure provides for other aspects and embodiments that will be apparent to one of skill in the art in light of the following description.

### DETAILED DESCRIPTION

In a general sense, the disclosure relates to a composition comprising an amount of copolymer-1 (also known as glatiramer, glatiramer acetate, or COPAXONE®) and an amount of an agent that has anesthetic properties. The inventor has identified that compositions comprising an effective amount of copolymer-1 and an amount of an anesthetic can reduce the frequency and/or the severity of Site Reactions that are associated with COPAXONE® therapy. The composition may be provided in a form that is suitable for administration to a subject having, or suspected of having, a disease or condition that can be treated using copolymer-1. Accordingly, the compositions disclosed herein enable methods for reducing or eliminating the Site Reactions associated with copolymer-1 (e.g., COPAXONE®) therapy, and can improve upon the existing methods and efficacies for treating patients afflicted with multiple sclerosis (e.g., RRMS).

All patents and non-patent literature references cited herein are incorporated by reference in their entirety.

It should be appreciated that all numerical ranges disclosed herein are intended to include any particular number within that range as well as sub-ranges that fall within the scope of the broader range. For example, a range of 0.01% to 5.0% will be understood to also encompass ranges falling at or above 0.01% and at or below 5.0% (e.g., 3.7%, 1.0%, 0.02%-0.04%, 0.02%-4.5%, 0.05%-4.08%, or 0.03%-1.0%, etc.). These are just examples of the types of numbers and ranges that would be encompassed.

### Site Reactions

The compositions and methods disclosed herein advantageously provide for the reduction in the frequency and/or the severity of one or more of the Site Reactions associated with the administration of copolymer-1 (e.g., glatiramer acetate and/or COPAXONE® therapy). Non-limiting examples of Site Reactions include, but are not limited to, skin reactions and injection site-specific reactions which may include, for example, residual lumps/masses (lipatrophy); induration; tenderness/soreness; pain; stinging; inflammation; cutaneous hypersensitivity; urticaria; edema; pruritis/itching; bruising; erythema; red spots; rashes; and welts, or any combinations thereof. In some embodiments the Site Reaction is selected from the group consisting of injection site tenderness, soreness, pain, stinging, skin reaction, and itching, or combinations thereof. In some embodiments the Site Reaction is selected from the group consisting of injection site pain and skin reaction (e.g. whether injection site-specific or not).

### Methods

In an aspect the disclosure provides a method for treating a disease or disorder that is responsive to treatment with copolymer-1 comprising administering to a subject in need thereof a pharmaceutically effective amount of a composition comprising copolymer-1 and a local anesthetic. In some embodiments the disease or disorder comprises multiple sclerosis such as, for example, RRMS. As used herein, “treating” means slowing or stopping the disease or slowing or stopping the progression of the disease. In some embodiments treating can mean remission of disease. In some embodiments, treating can also relate to alleviating or ameliorating one or more clinical indications or symptoms associated with the disease. In some embodiments, treating relates to reducing the frequency of disease relapses in patients with Relapsing-Remitting Multiple Sclerosis (RRMS).

In another aspect the disclosure provides a method for reducing the Site Reactions associated with administration of copolymer-1 (e.g., glatiramer acetate and/or COPAXONE® therapy) comprising administering to a subject in need thereof a composition disclosed herein. Suitably, Site Reactions are associated with methods that comprise an injection as the route of administration. As used herein “reducing” when used in connection with methods for “reducing Site Reactions” means that the method alleviates or ameliorates the severity of the clinical indication of the particular Site Reaction(s) (e.g., pain, itching etc.). The term may also relate to reducing the duration and/or the frequency of a particular Site Reaction. As will be appreciated, the method need not entirely eliminate the occurrence of the Site Reaction, and the efficacy of the method is typically determined by a qualitative assessment of the subject being treated (e.g., an assessment performed by a clinician, or a patient self-assessment). In some embodiments, the method is effective to substantially reduce the injection site pain associated with copolymer-1 therapy. In some embodiments the method is effective to substantially reduce the injection site pain associated with copolymer-1 therapy and further provides for a reduced adverse skin reaction. “Substantially reduce” in connection with the injection site pain associated with copolymer-1 therapy can mean that a qualitative assessment of the improvement in reduction of pain severity arising from the method (when compared to a method that does not comprise a composition as disclosed herein) is at least about 10% to about 100% (e.g., at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%). In embodiments, the method can essentially eliminate any pain associated with copolymer-1 therapy. In embodiments, the method can reduce the duration of any pain associated with copolymer-1 therapy (e.g., reduces duration of pain by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%).

In some embodiments, the methods described above are effective in treating a disease or disorder that is responsive to treatment with copolymer-1 (e.g., multiple sclerosis) and reducing the Site Reactions associated with the administration of copolymer-1 (e.g., pain, skin reactions, inflammation, itching, etc.).

In another aspect, the disclosure relates to a method for increasing patient compliance with a therapeutic regimen that comprises administration of copolymer-1 to the patient. In embodiments, the method comprises the administration of a composition as disclosed herein to the patient in need of treatment. The method can provide for the reduction of at least one or more Site Reaction(s) in a patient receiving a copolymer-1 therapy and induces the patient to comply with the therapeutic dosing schedule (e.g., daily or less frequent injections). Such compliance is important as failure to adhere to a therapeutic schedule may result in an exacerbation or relapse of disease (e.g., MS, RRMS) symptoms.

In any of the methods described herein, the method can comprise a step that includes separate administration of an additional amount of an anesthetic, prior to, following, or at the same time as the administration of the composition as described herein. In embodiments, the additional step can comprise an injectable local anesthetic. In embodiments, the additional step can comprise a topical local anesthetic. Suitably the additional amount of anesthetic is administered near or at the site of the amount of the composition comprising copolymer-1 and anesthetic.

### Compositions

In an aspect, the disclosure provides for a composition comprising an amount of copolymer-1 and an amount of a local anesthetic. In some embodiments, the composition can further comprise an amount of a carrier, diluent, vehicle, or adjuvant. In some embodiments, the carrier, diluent, vehicle, or adjuvant can be a pharmaceutically acceptable carrier, diluent, vehicle, or adjuvant. Suitably, the composition comprises an amount of copolymer-1 that is effective to treat a particular disease or clinical indication of the disease that is treatable by administration of copolymer-1 including, for example, multiple sclerosis and symptoms associated with multiple sclerosis. The amount of anesthetic in the composition is suitably an amount effective to alleviate or ameliorate the severity of a Site Reaction associated with the administration of copolymer-1, and/or reduce the frequency of the occurrence of a Site Reaction associated with administration of copolymer-1.

### Copolymer-1.

As used herein, “copolymer-1,” “glatiramer,” “glatiramer acetate,” or “COPAXONE®” are generally interchangeable with each other and refer to a mixture of polypeptides as generally disclosed in U.S. Pat. No. 3,849,550, where the mixture comprises alanine, glutamic acid, lysine, and tyrosine in a molar ratio of approximately 6:2:5:1, respectively. Copolymer-1 can be synthesized by chemically polymerizing the four amino acids, forming products having broad ranges of average molecular weight peptides (e.g., from 2,500 daltons to 40,000 daltons). The products can be further processed, or alternatively synthesized, in order to create polypeptide products having lower average molecular weights, as further discussed below. While these different terms are generally interchangeably used to refer to the same active agent polypeptide, it should be appreciated that the terms “copolymer-1,” “glatiramer,” and “glatiramer acetate” refer to the generic name of the active polypeptide mixture, while “COPAXONE®” is used typically to refer to the proprietary name of the FDA-approved drug. In embodiments, the compositions comprise an injectable form of copolymer-1 (e.g., a sterile solution of glatiramer, glatiramer acetate, or COPAXONE®).

In some embodiments, copolymer-1 may be substantially free of species of copolymer-1 having a molecular weight of over 40 kilodaltons (KDa). In some embodiments, the copolymer-1 may contain less than 5% of species of copolymer-1 having a molecular weight of 40 KDa or more. In further embodiments, the copolymer-1 may contain less than 2.5% of species of copolymer-1 having a molecular weight of 40 KDa, or more. In some embodiments, copolymer-1 may be substantially free of species of copolymer-1 having a molecular weight of over 20 KDa (e.g., may contain less that 5% or less than 2.5% of species of copolymer-1 having a molecular weight of 20 KDa or more).

In some embodiments, copolymer-1 can comprise over 75% of its molar fraction within the molecular weight range from about 2 KDa to about 20 KDa. In some embodiments, copolymer-1 may comprise an average molecular weight of about 4 to about 8.6 KDa. In further embodiments, copolymer-1 can comprise an average molecular weight of about 4 to about 9 KDa, which is inclusive of copolymer-1 having an average molecular weight of about 6.25 to about 8.4 KDa. In some embodiments the average molecular weight of copolymer-1 is about 5 KDa to about 9 KDa. In further embodiments, copolymer-1 is provided as COPAXONE®.

Methods for preparing copolymer-1 are known in the art and are disclosed, for example, in U.S. Pat. Nos. 3,849,550; 5,800,808; 5,981,589; 6,048,898; 6,054,430; 6,342,476; 6,362,161; 6,620,847; 6,939,539; 7,199,098; and 7,625,861, and U.S. Published Patent Application Nos. U.S. 2006/0172942; U.S. 2006/0154862; U.S. 2007/0141663; U.S. 2010/0036092; U.S. 2010/0324265; U.S. 2012/0077754 and U.S. 2012/0123094. In some embodiments, the N-carboxyanhydrides of tyrosine, alanine, gamma-benzyl glutamate and eta-N-trifluoroacetyllysine are polymerised at ambient temperature in anhydrous dioxane with diethylamine as initiator. The deblocking of the gamma-carboxyl group of the glutamic acid is effected by hydrogen bromide in glacial acetic acid and is followed by the removal of the trifluoroacetyl groups from the lysine residues by 1 M piperidine. For the purposes of these synthetic methods, the terms “ambient temperature” and “room temperature” should be understood to mean a temperature ranging from about 20° C. to about 26° C.

Accordingly, copolymer-1 with the desired and/or required molecular weight profile can be obtained by any methods known in the art. Such methods include, for example, dialysis, ultrafiltration, size-exclusion chromatography, FPLC, and HPLC of copolymer-1 mixtures containing high molecular weight species and collecting the dialysate, filtrate, and/or fractions without the undesired species. Alternatively, partial acid or enzymatic hydrolysis may be used to remove the high molecular weight species with subsequent purification by dialysis, ultrafiltration, or chromatography (size-exclusion, HPLC, HIC, FPLC, etc.). A further method that can be used to obtain copolymer-1 with the desired molecular weight profile is by preparing the desired species while the amino acids are still protected and obtain the correct species directly upon removal of the protecting groups.

Suitably, the amount of copolymer-1 in the compositions disclosed herein is effective to treat a disease or disorder, or one or more related clinical indication(s) of a disease or disorder, in which copolymer-1 has been shown to be effective. To date, copolymer-1 (as COPAXONE®) is approved for daily administration (subcutaneous injection) to patients suffering from multiple sclerosis in a daily dose of 20 mg. Disclosure of various copolymer-1 formulations as well as COPAXONE® are provided in U.S. Pat. No. 5,800,808; U.S. Pat. No. 5,981,589; U.S. Pat. No. 6,048,898; U.S. Pat. No. 6,054,430; U.S. Pat. No. 6,342,476; U.S. Pat. No. 6,362,161; U.S. Pat. No. 6,620,847; U.S. Pat. No. 6,939,539; and U.S. Pat. No. 7,199,098. European Patent EP2275086, as well as U.S. 2011/0060279 and U.S. 2011/0066112, relate to a reduced volume formulation (0.5 mL unit dose of 20 mg glatiramer acetate) and related methods and devices for administration, which is encompassed by embodiments of this disclosure. In some embodiments, the compositions disclosed herein may comprise an amount of copolymer-1 that is sufficient for administration to a subject (e.g., a subject suffering from multiple sclerosis) at a dosage of 40 mg and allows for injections every other day, or three times per week. Examples of such low frequency formulations are disclosed in U.S. Pat. No. 8,232,250 and U.S. 2011/0046065.

The particular amount of copolymer-1 and/or the particular volume of the copolymer-1 dosage are not critical to the instant disclosure, as long as the composition includes a dosage of copolymer-1 (and is administered to a patient in a way) that provides a clinical benefit. In some embodiments the amount of copolymer-1 in the composition comprises, on a unit dosage basis, about 5 mg to about 100 mg. In embodiments, the amount of copolymer-1 in a unit dosage may be 10 to 80 mg; or 12 to 70 mg; or 14 to 60 mg; or 16 to 50 mg; or 18 to 40 mg; or 20 to 30 mg; or 20 mg. Alternatively, the amount of copolymer-1 administered to a subject may be in the range from 10 to 600 mg/week; or 100 to 550 mg/week; or 150 to 500 mg/week; or 200 to 450 mg/week; or 250 to 400 mg/week; or 300 to 350 mg/week; or 300 mg/week. In other embodiments, the total amount of copolymer-1 administered to a subject may be in the range from 50 to 150 mg/day; or 60 to 140 mg/day; or 70 to 130 mg/day; or 80 to 120 mg/day; or 90 to 110 mg/day; or 100 mg/day. In further embodiments, the total amount of copolymer-1 administered to a subject may be in the range from 10 to 80 mg/day; or 12 to 70 mg/day; or 14 to 60 mg/day; or 16 to 50 mg/day; or 18 to 40, mg/day; or 19 to 30 mg/day; or 20 mg/day.

Thus, in embodiments the amount of copolymer-1 in a unit dose can be about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg or more. The volume of unit dosage for an injectable composition can vary widely based on any number of factors such as, for example, the amount and solubility of copolymer-1, amount and solubility of anesthetic, presence/amount of a carrier (e.g., mannitol), vehicle, adjuvant, buffer component (e.g., sterile isotonic aqueous buffer), or osmotic component, and the like. In some embodiments the composition can be formulated for injection (e.g., subcutaneous, intravenous, intramuscular, intraperitoneal, depot, etc.) and can comprise a unit dosage volume of about less than 0.1 mL to about 5.0 mL or more (e.g., 5.0 mL, 4.5 mL, 4.0 mL, 3.5 mL, 3.0 mL, 2.5 mL, 2.0 mL, 1.9 mL, 1.8 mL, 1.7 mL, 1.6 mL, 1.5 mL, 1.4 mL, 1.3 mL, 1.2 mL, 1.1 mL, 1.0 mL, 0.9 mL, 0.8 mL, 0.7 mL, 0.6 mL, 0.5 mL, 0.4 mL, 0.3 mL, 0.2 mL, or about 0.1 mL or less).

Accordingly, one of skill in the art will appreciate that the compositions and methods described herein can be applied to any formulation, particularly liquid solutions, that includes copolymer-1 such as, for example, combination therapies, low volume injection, low frequency injection, low volume and low frequency injection, including those yet to be developed (e.g., sustained release, controlled release) and provides a particular benefit for injectable formulations. Thus, the scope of the disclosure covers compositions comprising copolymer-1 and an anesthetic each in effective amounts and in relative ratios as discussed herein.

### Anesthetics.

An “anesthetic” as used herein means an agent (e.g., a drug) that causes a reversible and/or temporary loss of sensation. Anesthetics are different from painkillers (analgesics) which relieve pain without eliminating sensation. Anesthetics encompass a wide variety of drugs and are often administered in order to facilitate surgery as well as dental and minimally invasive procedures. Anesthetics are often classified as either a general anesthetic or a local anesthetic. General anesthetics cause a reversible loss of consciousness, while local anesthetics cause a reversible loss of sensation over a limited region of the body, typically while maintaining consciousness. Suitably, the anesthetic used in connection with the compositions described herein possess one or more of the following characteristics: (i) it is generally inert when combined with other active agents; (ii) it has a long duration of action; (iii) it lacks toxicity or has a relatively low toxicity; and (iv) it has been used without the occurrence of any side effects. Further, the anesthetic is suitably soluble and stable in a solution that is also able to maintain the solubility of copolymer-1 (e.g., does not form precipitate).

In some embodiments, the compositions disclosed herein can comprise a local anesthetic. As noted above, local anesthetics are agents that prevent transmission of nerve impulses without causing unconsciousness. Structurally, local anesthetics typically include an aromatic group and an amine moiety linked by an intermediate carbon or carbon and heteroatom chain. They are often classified into groups as ester- or amide-based compounds. Some non-limiting examples of local anesthetics include procaine, amethocaine, cocaine, lidocaine, prilocaine, bupivacaine, levobupivacaine, ropivacaine, chloroprocaine, mepivacaine, and dibucaine.

Amide-based local anesthetics (e.g., articaine, novocain (procaine), chloroprocaine (Nesacaine), lidocaine (Xylocaine), prilocaine (Citanest), etidiocaine (Duranest), bupivicaine (Marcaine), levobupivacaine, ropivacaine (Naropin), mepivacaine (Polocaine, Carbocaine), and dibucaine) are generally heat-stable, and have a long shelf life (around 2 years), and as such, amide-based local anesthetics are particularly suitable for use in the compositions and methods described herein. In some embodiments, a local anesthetic can optionally be combined with an amount of epinephrine. Typically, the amide-based local anesthetics are usually racemic mixtures and have a slower onset and longer half-life than ester-based anesthetics. Two specific exceptions include levobupivacaine (which is S(-)-bupivacaine) and ropivacaine (S(-)-ropivacaine). The amide-based local anesthetic agents are generally used within regional and epidural or spinal techniques, due to their longer duration of action, which provides adequate analgesia for procedures including surgery, child labor, and symptomatic relief. In some embodiments, the local anesthetics are in a form suitable for administration my injection (e.g., subcutaneous injection). As will be appreciated by those skilled in the art, local anesthetics that are administered by intrathecal injection must be preservative-free, and are often administered at lower dosages.

Local anesthetics can block nerve transmission as well as exhibit anti-inflammatory properties. Certain membrane receptors such as PGE2 EP1 receptor, for example, are involved with inflammation and pain, and can be affected by local anesthetics. Accordingly, in some embodiments the local anesthetic can be selected from those agents that can bind to receptor proteins that are involved in biochemical pathways associated with inflammation and/or pain (e.g., PGE2 EP1 receptor). (See, Honemann, C. W., et al., Anesth Analg. (2001) 93:628-634.). Thus, in some embodiments the composition may comprise a concentration of local anesthetic that is lower than concentrations currently used in clinical practice, providing for a reduced Site Reaction and a decreased risk of toxicity. (See, Rosenberg, P. H., et al., Regional Anesthesia and Pain Medicine (Nov-Dec 2004) 29(6):564-575).

In some embodiments the composition may comprise a local anesthetic that is effective to reduce a Site Reaction without substantially adding to the volume of composition that is required per dose. In some embodiments, the amount of local anesthetic in the composition is about 0.01% to about 50% (w/v), or more typically about 0.01% to about 10% (e.g., 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10.0%, etc.) depending on factors such as the recommended highest dosage and the efficacy of the particular local anesthetic.

In embodiments the amount of copolymer-1 and anesthetic provides a composition for injection having a volume of 1.5 mL or less (e.g., 1.4 mL or less, 1.3 mL or less, 1.2 mL or less, 1.1 mL or less, 1.0 mL or less, 0.9 mL or less, 0.8 mL or less, 0.7 mL or less, 0.6 mL or less, 0.5 mL or less, 0.4 mL or less, 0.3 mL or less, 0.2 mL or less, or about 0.1 mL or less). Typically, the volume will range from about 1.5 mL to about a minimum volume of about 0.1 mL. In some embodiments, the local anesthetic is preferably formulated with copolymer-1 to provide a composition that contains the minimum amount of local anesthetic needed to reduce a Site Reaction, and provide for the lowest possible injection volume in order to provide an effective amount of copolymer-1. In such embodiments, the composition may contain a reduced injection volume of glatiramer acetate (e.g., 20 mg per 0.5 mL injection) in a daily dose, and/or an increased dose of glatiramer acetate (40 mg per 1.0 mL or 0.5 mL injection) in a low-frequency dose (relative to the FDA approved COPAXONE® product (20 mg per 1.0 mL injection), such as described herein.

In some embodiments, the anesthetic comprises at least one local anesthetic selected from the group including, but not limited to, procaine, etidocaine, tetracaine, lidocaine, prilocaine, bupivicaine, levobupivacaine, ropivacaine, mepivacaine, chloroprocaine and dibucaine, or any combination thereof. In further embodiments, the anesthetic comprises lidocaine, chloroprocaine, mepivacaine, ropivacaine, or bupivicaine. In some embodiments, the anesthetic comprises lidocaine, chloroprocaine, or mepivacaine. In some embodiments the anesthetic comprises bupivacaine. In some embodiments the anesthetic comprises ropivacaine. As will be appreciated by one of skill in the art, certain local anesthetics can be provided as solutions that contain an optional preservative. As some preservatives (e.g., methylparaben, sodium bisulfate, etc.) may cause allergic reaction, it may be preferable to use the local anesthetic in preservative-free form, even though the preservative should have no impact on the efficacy of the composition to reduce Site Reactions. General guidelines for anesthetic dosage ranges are known in the art. (See, Rosenberg, P. H., et al. 2004).

While a number of synthetic routes as well as routes for isolating and purifying anesthetics are well known in the art, the anesthetics provided herein are suitably available from commercial sources and/or can be packaged with the core drug for use by a patient according to instructions provided by a manufacturer of the core drug and/or a treating physician.

The compositions described herein may be formulated by conventional methods known in the art. In some embodiments, the copolymer-1 portion of the composition can be lyophilized and formed into an aqueous solution suitable for subcutaneous injection. The anesthetic can be added to the copolymer-1 fraction (or vice-versa) at any time (e.g., added to the copolymer-1 lyophilisate, a solution of copolymer-1, or to the COPAXONE® drug product) or formulated in a step-wise manner.

As discussed herein, the amount of anesthetic included in the composition may be determined by one of skill in the art using methods and information well known in the art. Relevant considerations include the particular anesthetic to be used, the route of administration of the composition, the concentration of the anesthetic preparation, dilution percentages of vascoconstrictors (if included), maximum recommended doses, patient weight, height, sex, health status, disease stage, severity of symptoms, other medications taken by the patient, and the like. In embodiments, the amount of the anesthetic is effective to provide relief of the particular Site Reactions to be treated (e.g., reduce the severity of one or more clinical indication of the Site Reaction, reduce the frequency of one or more clinical indication of the Site Reactions, or both). Recommended dosages for local anesthetics are well known in the art. (See, Rosenberg, P. H., et al. (2004)). In some embodiments the dosage of the anesthetic may be lower than the typical recommended dosage guideline. For example, a local anesthetic dosage may be determined based on the amount required to block one or more receptors involved in biochemical pathways associated with inflammation, prostaglandin synthesis, and/or pain, and which can be anywhere from about two- to about ten- or twenty-fold less than dosages used for neural blockade in clinical practice. (See, Pietruck, C., et al., Anesth Analg. (2003) 96:1397-1402). Non-limiting examples of such concentration ranges may include about 0.01% to 1.0% anesthetic (w/v) (e.g., 0.02%-0.04%, or 0.05%-0.08%, or 0.03%-1.0%, etc.).

In some embodiments, the effective amount of a local anesthetic may be determined based on a controlled titration dosing trial (e.g., from a lower concentration to higher concentration) where the amount of local anesthetic required to provide relief to a specific subject is determined by feedback from the subject (e.g., patient follow up reporting of reduced or ameliorate site reaction(s)). In such embodiments, one of skill in the art may determine an effective amount of a given anesthetic by starting administration with a composition comprising a low concentration of anesthetic and, based on patient feedback, can maintain or incrementally increase the concentration of anesthetic as needed in order to provide relief of the site reaction to the specific individual. This method of titrating to an effective amount can provide for finely-tuned dosages for individual local anesthetics, and for individual patients. One of skill in the art will be able to adjust and optimize the amount of anesthetic in the compositions based on the considerations discussed herein (e.g., the particular anesthetic used, the general health of the patient, the severity of Site Reactions, the amount of copolymer-1, individual response to an anesthetic, etc.).

In some embodiments, the composition comprises copolymer-1 and an anesthetic selected from chloroprocaine, lidocaine, and mepivacaine. In further embodiments, the composition comprises a total amount of chloroprocaine, lidocaine, or mepivacaine active ingredient of about 0.1% to about 1.0% (w/v).

In some embodiments the composition comprises an amount of copolymer-1 (as 20 mg daily dosage) and an amount of chloroprocaine, lidocaine, or mepivacaine (from about 0.1% to about 1.0% (w/v) in solution). In some embodiments, the composition is formulated as an injectable solution. In some embodiments, the chloroprocaine, lidocaine, or mepivacaine is provided as a stock solution ranging in concentration from 1.0%-3.0%, and is added to the copolymer-1 solution to an appropriate dilution concentration (e.g., less than about 1.0% and greater than about 0.1%).

In one embodiment, the composition comprises copolymer-1 and bupivacaine. In another embodiment, the composition comprises copolymer-1 and ropivacaine. In another embodiment, the composition comprises copolymer-1 and a combination of bupivacaine and ropivacaine. In further embodiments, the composition comprises a total amount of bupivacaine or ropivacaine active ingredient of about 0.01% to about 1.0% (w/v).

In some embodiments the composition comprises an amount of copolymer-1 (as 20 mg daily dosage) and an amount of bupivacaine or ropivacaine (from about 0.01% to about 1.0% (w/v) in solution). In some embodiments, the composition is formulated as an injectable solution. In some embodiments, the bupivacaine or ropivacaine is provided as a stock solution ranging in concentration from 0.25%-0.75%, and is added to the copolymer-1 solution to an appropriate dilution concentration (e.g., less than about 1.0% and greater than about 0.01%). In some embodiments, the amount of copolymer-1 can be increased to about 40 mg or more per dose, such that the frequency of injection can be reduced from daily to every other day or even less frequently (e.g., 2 or 3 doses/week). When the composition comprises copolymer-1 dosages above 20 mg/dose (e.g., 25, 30, 35, 40, 45, 50, 55, 60, or more mg/dose), some embodiments provide for the amount of anesthetic in the composition to be increased relative to the amount (or ratio of copolymer-1:anesthetic) contained in a composition comprising a 20 mg dose of copolymer-1 in order to achieve the same effect regarding reduction of a Site Reaction. Thus, in some embodiments the composition comprises a ratio of copolymer-1 to local anesthetic ranging from about 20:0.01 to about 1:1 (w/w).

In some embodiments, the composition can comprise further purification and/or polishing steps such as using sterile filtration, HPLC, or the like in order to remove particulates and/or impurities and the like.

In further embodiments, the composition can comprise a combination of active agents (combination therapy) that includes an anesthetic, an amount of glatiramer, and one or more additional active agents, such as an agent used in the treatment of multiple sclerosis or a related condition or disorder. The additional active agents can be selected from any that have been combined, or that can be combined, with copolymer-1 including non-limiting examples of (i) active protein and polypeptide agents (e.g., U.S. 2012/0087934, U.S. 2012/0010148, U.S. 2011/0044945); (ii) antibodies and active fragments thereof (e.g., U.S. 2012/0199516); and (iii) small molecule agents (e.g., U.S. 2012/0027718, U.S. 2011/0268699). In such combination compositions, the anesthetic is selected and added in an amount that allows for the reduction of a site reaction, while also not adversely reacting with either the copolymer-1 or the additional active agent(s).

The compositions provided herein may be suitably formulated as pharmaceutical compositions for administration to a subject. In such embodiments, the pharmaceutical compositions comprise copolymer-1, an anesthetic, and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Non-limiting examples of pharmaceutically acceptable carriers include one or more of water, saline, phosphate buffered saline, dextrose, glycerol, ethanol and the like, as well as combinations thereof. Such compositions may also include isotonic agents such as, for example, sugars, polyalcohols (e.g., mannitol, sorbitol, or sodium chloride, etc.). In such embodiments, the compositions can include pharmaceutically acceptable auxiliary substances such as wetting or emulsifying agents, preservatives or buffers, which can enhance the shelf life or effectiveness of the composition.

While the compositions described herein may be provided in any known and suitable form (e.g., liquid, semi-solid and solid dosage forms, such as injectable and infusible solutions, dispersions, suspensions, tablets, pills, powders, liposomes and suppositories, etc.), the compositions are typically provided in the form of solutions suitable for injection. Thus, the methods and compositions encompassed by the disclosure typically include a parenteral administration route (e.g., intravenous, subcutaneous, intraperitoneal, intramuscular). In embodiments, the composition is administered by subcutaneous injection. Standard methods for the preparation and use of such injectable compositions can be used with the compositions and methods described herein. See, e.g., Remington, The Science and Practice of Pharmacy, 21st Edition, 2005, (e.g., Chapters 41-42), incorporated herein by reference.

The Examples that follow are intended to be merely illustrative of the aspects and embodiments described above and should not be viewed as limiting to the scope of the appended claims.

### EXAMPLES

### Example 1. Composition Including Copolymer-1 and Anesthetic

A series of compositions comprising an amount of copolymer-1 and a local anesthetic were generated in order to provide for a formulation that was substantially free of Site Reactions upon injection. Briefly, the methodology involved titrating amounts of an injectable local anesthetic (bupivacaine, 0.5%) to a solution of copolymer-1 (provided as COPAXONE®, 20 mg injectable syringe). Addition of 0.2 mL of 0.5% bupivacaine solution to 1.0 mL of COPAXONE® provided for a composition that includes the approved daily dose of copolymer-1 (20 mg) and a concentration of bupivacaine of 0.083% that reduces Site Reactions. This percentage of local anesthetic is well below clinical dosages typically used for skin infiltration for pain control (e.g. 0.25%). The composition was stable for over 30 days both at room temperature and when refrigerated.

### Example 2. Efficacy of Pain Reduction of Copolymer-1 and Bupivacaine Composition

The composition described in Example 1 (0.2 mL of bupivacaine (at 0.5%) added to 1.0 mL COPAXONE®, 20 mg injectable syringe) was used in this Example.

The inventor, Dr. Robbins, an anesthesiologist and pain management specialist familiar with the use of local anesthetics in procedures such as spinal epidurals, has been diagnosed with multiple sclerosis and has been prescribed COPAXONE® in order to treat the disease. After complying with the therapy for about five months, the pain associated with the treatment (skin wheals, immediate pain upon injection and continuing post-injection pain) affected compliance with his prescribed injection schedule and he discontinued use of the drug. Oral pain management (Motrin) and immediate icing of the injection site were ineffective in alleviating the pain associated with the COPAXONE® injection. In an effort to continue therapy with COPAXONE®, the inventor began investigation of alternative methods of reducing or eliminating the Site Reactions resulting from the daily injections associated with treatment using Copaxone®. As an adjunct to the copolymer-1 therapy, the inventor explored use of various anesthetics. Through these studies he identified that a combination formulation of COPAXONE® and bupivacaine, as described in Example 1, was particularly effective in reducing the Site Reactions he was experiencing daily while using COPAXONE® alone.

Upon continued administration of the bupivacaine and COPAXONE® composition over a period of seven months the Site Reactions have been essentially eliminated. The formulation comprising a combination of COPAXONE® and bupivacaine is about 90%-98% pain-free upon injection, and skin reactions are minimally observable. Thus, the inventor has demonstrated in a prolonged seven month study of daily injections that the addition of bupivacaine to the COPAXONE® formulation provides this result at a concentration of about 0.083% (which provides greater than 95% inhibition of 5-hydroxytryptamine (5-HT) induced plasma extravasation; see, e.g., Pietruck, C., et al., Anesth Analg. (2003) May; 96(5):1397-1402.). Unexpectedly, this dosage is threefold less than doses used clinically for axonal blocking effects as reported in the literature.

Previous attempts to use oral antihistamines to reduce local injection site reactions (itching, pain, swelling, redness) that arise from glatiramer acetate injection have been documented as unsuccessful (Pardo, G. et al., J. Neuroscience Nursing (2010) February; 42(1):40-46). Accordingly, the formulations described and exemplified herein that comprise a glatiramer acetate injection (modified COPAXONE®) combined with a local anesthetic provide for the reduction of similar side effects, or Site Reactions, as disclosed above.

### Further Compositions

Additional compositions comprising an amount of copolymer-1 and a local anesthetic were generated in order to provide for a formulation that was substantially free of Site Reactions upon injection. Briefly, a similar methodology as described above for the bupivacaine anesthetic was followed for the injectable local anesthetics ropivacaine (0.5% stock), lidocaine (2.0% stock), chloroprocaine (2.0% stock), mepivacaine (2.0% stock). These stock solutions were added to a solution of copolymer-1 (provided as COPAXONE®, 20 mg injectable syringe) to provide final concentrations of 0.05%-0.08% for ropivacaine and up to a final concentration of 0.33% each of lidocaine, mepivacaine, and chloroprocaine. While some relief was experienced at the relatively high concentrations (0.33%) of lidocaine, mepivacaine, and chloroprocaine, only the ropivacaine and bupivacaine compositions were effective to provide Site Reaction relief at low dosages (e.g., about 0.05% to 0.08%). It is contemplated that higher concentrations (e.g., about 0.66%) of lidocaine, mepivacaine, and chloroprocaine may provide relief of Site Reactions that approaches the relief provided by the ropivacaine and bupivacaine compositions.

### Example 3. Prophetic Clinical Evaluation of Copolymer-1 and Bupivacaine Composition

A broader evaluation (e.g., multicenter, randomized, double-masked, vehicle-controlled, parallel-group study or single center, randomized, investigator-masked, paired-comparison clinical trial, etc.) of the compositions disclosed herein can be carried out using, for example, the compositions of Examples 1 and 2. The study subjects will include a number of patients suffering from multiple sclerosis and will be divided among equal sized treatment groups. Various inclusion criteria for the study will be established and may include having COPAXONE® prescribed as a therapy. The patient demographics will be tracked and the treatment groups will be constructed such that there will be no significant demographic differences between treatment groups.

One group of treatment subjects will receive COPAXONE® while the other group of treatment subjects will receive the composition of Examples 1 and 2 (e.g., COPAXONE® plus bupivacaine). The subjects will be allowed to take an analgesic or use modalities such as the application of an ice pack as needed for pain, and will undergo regular physical examinations. The patients will report and/or record any Site Reactions (e.g., pain, itching, etc.) they experience and will provide a qualitative assessment of the degree (e.g., no pain, mild, moderate, severe, intolerable, visible, not visible, etc.). The data and results from the study will be evaluated in order to determine differences between the treatment groups.

It is expected that patients receiving the composition comprising COPAXONE® and bupivacaine or other injectable local anesthetics will experience reduction in the Site Reactions observed in the patients receiving COPAXONE® alone. It is further expected that the patients receiving the COPAXONE® and bupivacaine (or ropivacaine, lidocaine, chloroprocaine, or mepivacaine) composition will also not exhibit any reduction in therapeutic efficacy in the management of the multiple sclerosis. It is also expected that a higher incidence of patient compliance with the therapeutic regimen will be observed for those patients receiving the COPAXONE® and bupivacaine (or ropivacaine, lidocaine, chloroprocaine, or mepivacaine) composition.

1. (canceled)

2. A composition comprising copolymer-1; a local anesthetic; and a carrier, diluent, or vehicle.

3. The composition of claim 2, wherein the local anesthetic is selected from the group consisting of articaine, novocain, procaine, tetracaine, chloroprocaine, lidocaine, prilocaine, etidiocaine, bupivicaine, levobupivacaine, ropivacaine, mepivacaine, and dibucaine, or any combination thereof.

4. The composition of claim 2, wherein the anesthetic is selected from the group consisting of bupivacaine and ropivacaine.

5. The composition of claim 2, wherein the anesthetic is selected from the group consisting of lidocaine, chloroprocaine, and mepivacaine.

6. The composition of claim 2, wherein the copolymer-1 comprises injectable glatiramer acetate.

7. The composition of claim 2, wherein the copolymer-1 comprises COPAXONE®.

8. The composition of claim 2, wherein the amount of local anesthetic in said composition is about 0.01% to about 50% (w/v).

9. The composition of claim 4, wherein the total amount of bupivacaine or ropivacaine in the composition is about 0.01% to about 1.0% (w/v).

10. The composition of claim 4, wherein the composition comprises a ratio of copolymer-1 to bupivacaine or ropivacaine of about 20:0.01 to about 1:1 (w/w).

11. The composition of claim 10, wherein the ratio of copolymer-1 to bupivacaine or ropivacaine in the composition is about 40:1 to about 10:1 (w/w).

12. The composition of claim 10, wherein the ratio of copolymer-1 to bupivacaine or ropivacaine in the composition is about 20:1 (w/w).

13. The composition of clam 2, wherein the amount of copolymer-1 is from about 5 mg to about 60 mg per dose.

14. The composition of claim 13, wherein the composition is provided as a unit dose of 0.1 mL to about 1.5 mL.

15. The composition of claim 2, wherein the anesthetic is included in an amount that is effective to reduce the incidence and/or severity of at least one Site Reaction associated with copolymer-1 therapy, wherein the at least one Site Reaction is selected from the group consisting of: lipatrophy, injection site tenderness, injection site pain, injection site stinging, general pain, itching, skin reaction, bruising, formation of red spots, skin welts, and skin wheals.

16. A method for treating multiple sclerosis comprising administering a composition according to claim 2 to a subject in need thereof.

17. A method for reducing a Site Reaction associated with administration of copolymer-1 to a subject, comprising administering to a subject in need thereof a composition according to claim 2.

18. The method of claim 17, wherein the Site Reaction is selected from the group consisting of injection site lipatrophy, injection site tenderness, injection site pain, injection site stinging, general pain, itching, skin reaction, bruising, formation of red spots, skin welts, and skin wheals.

19. A method for increasing patient compliance with a therapeutic regimen that comprises administration of copolymer-1, wherein the method comprises administering to the patient a composition according to claim 2.

20. The composition according to claim 2 wherein the copolymer-1 composition comprises a mixture of polypeptides composed of glutamic acid, lysine, alanine and tyrosine, wherein the mixture has an average molecular weight of about 4 kilodaltons to about 9 kilodaltons, wherein the mixture of polypeptides is non-uniform with respect to molecular weight and sequence.
